(19) United States
(12) Patent Application Publication
(10) Pub. No.: US 2004/0092589 A1
(43) Pub. Date: May 13, 2004

(54) Use of retinoic acid for treatment of autism

(76) Inventor: Liisa Neumann, Gambrills, MD (US)

Correspondence Address: Law Offices of Jerome J. Norris, Suite 305, 1901 Pennsylvania Avenue, N.W., Washington, DC, 20006, US

(21) Appl. No.: 10/175,919

(22) Filed: Jun. 21, 2002

(51) Int. Cl. A61K 31/203

(52) U.S. Cl. 514/559

(57) ABSTRACT

A method of treating a patient with autism to lessen or alleviate associated symptoms of deficient short term memory, inadequate independent problem solving skills, deficient spontaneous verbalizations, utterances of diminished length, infrequent request to interact with family members and friends, insufficient capacity to master complexity of play schemes, and a tendency toward abnormal emotional responses, comprising:

administering to the patient a therapeutically effective amount of retinoic acid to lessen or alleviate the symptoms.

### BACKGROUND OF THE INVENTION

1. Field of Invention

The invention relates to the use of retinoic acid to relieve some of the symptoms associated with autism; and more particularly, to the use of 13-cis-retinoic acid, also known as isotreinoin, to lessen or eliminate learning obstacles of subjective centered absorption that typically characterizes autistic subjects.

2. The Prior Art

Autism is a developmental disability which involves difficulties in language, behavior, and/or social skills. It is a spectrum disorder—meaning that it affects different people differently, in that some may have speech but unusual behaviors, whereas others may have no speech. Less severe cases may be diagnosed with Pervasive Developmental Disorder (PDD) or with Asperger's Syndrome (normal speech, but other behavioral/social problems).

Autism is a lifelong disability, meaning that if left untreated it will affect people their entire lives. Left untreated, many people with autism will not learn to talk, behave normally, or develop social skills to enable them to live on their own. To date, there is no one cure for autism; however, there are a wide variety of treatment options which work to varying degrees of success for some people.

The most accurate statistics on autism come from California, because they have a centralized reporting system of all diagnoses of autism. Their data shows that the incidence of autism is rising rapidly, increasing 258% in the last five years, and 36% in 1999 alone. Currently about 1 in 258 children in California is diagnosed with autisim (according to the strict DSM-IV criteria). Similarly, in Arizona the Department of Developmental Disabilities served 633 people with autism, and only 3 years later in 1999 it served 1057 people with autism, a dramatic increase.

It is not known why the increase is occurring, but there are several hypotheses. The cause(s) of autism is not known, but there is increasing evidence that many cases involve fungal and/or bacterial invasion/attack of the gut, which may limit the ability of the body to extract the right nutrients from food, and may allow some unwanted substances to enter the bloodstream. It is suspected that early use of oral antibiotics and some vaccinations may cause or contribute to these fungal and bacterial infections. Another possible cause is the “stealth virus”, which is difficult to detect. Biological approaches may help treat some of these causes, and therapy approaches may help improve behavior and communication.

Apparently, there is a genetic link to autism; if parents have one child with autism, there is a substantial chance (around 5-10%) that any other child will have autism. In fraternal twins, if one twin has autism, there is a 25% chance that the other twin will. In identical twins, if one twin has autism, there is a 95% chance that the other will.

Typical therapy approaches for individuals diagnosed with autism include: respite habilitation, speech therapy, occupational therapy and special education.

Some biological approaches that are known to have been used in treating subjects with autism are shown in the patents hereinafter described.

A method of treating autism is disclosed in U.S. Pat. No. 5,008,251. The method comprises administering to a patient at least about 1 mg/kg/day of a drug selected from the group consisting of AICA riboside, AICA ribotide, ribavirin and ribavirin monophospate.

U.S. Pat. No. 6,020,310 disclose a method for assisting in differential diagnosis and treatment of autistic syndromes comprising administering to the individual an effective amount of secretin, so that one or more symptoms of autistic disorder are improved.

A method of using secretin for treating autism is disclosed in U.S. Pat. No. 6,197,746 B1. The method comprises transdermally administering an effective amount of secretin to the individual, wherein one or more symptoms of autistic disorder are improved.

U.S. Pat. No. 6,365,593 B2 disclose the use of methylxanthines in the diagnosis and treatment of autistic disorder. More specifically, the method comprises:

obtaining a sample of urine from the individual;

measuring a level of a methylxanthine (MX) in the urine sample; and

comparing the level to a normal control or to a threshold level;

wherein a level below the normal control or below a threshold level of about 5.3 micrograms of methylxanthine/ml of urine indicates a possibility of autistic disorder.

There is a need to provide means for improving the education of special needs subjects with autism to lessen or alleviate the learning obstacle of subjective self-centered absorption.

There is a further need to provide orally administered means to enable an autistic subject to gain improvement in short term memory, improvement in independent problem solving skills, an increase in spontaneous verbalizations, an increase in the length of utterances, an increase in the frequency of requests to interact with family members and friends, an increase in ability to master the complexity of play schemes, and improvement toward more normalized emotional responses (e.g. not laughing when someone is seriously hurt in a movie, offering assistance to family members when they are hurt, and laughing along with siblings while watching cartoons instead of tensing and being overly excited.)

### SUMMARY OF THE INVENTION

One object of the present invention is to provide a method for treating an individual exhibiting one or more symptoms of autistic disorder with a therapeutically effective amount of retinoic acid to relieve or improve one or more of the symptoms of the disorder.

Another object of the present invention is to provide a therapeutically effective oral dosage of retinoic acid to alleviate learning obstacles in autistic subjects, who tend to to be characterized by subjective self-centered absorption.

A further object of the present invention is to provide a therapeutically effective oral dosage of retinoic acid to affect improvement in short term memory of autistic subjects, improvement in independent problem solving skills, an increase in spontaneous verbalizations, an increase in the length of utterances, an increase in the frequency of request necessary to interact with family members and friends, an increase in the ability to master the complexity of play schemes, and an increase toward more normalized emotional responses.

### DETAILED DESCRIPTION OF PREFERRED EMBODIMENT OF THE INVENTION

In the practice of the present invention, a therapeutically effective dose or retinoic acid is orally administered to a subject with autistic disorder. This disabling neurological disorder affects thousands of Americans and includes a number of subtypes, with many putative causes and relatively few treatments that tend to alleviate symptoms of the disorder. The disorder of the autistic spectrum may be present at birth or may have a subsequent onset; however, there are no clear cut biological markers for autism. In fact, diagnosis of the disorder is generally made by considering the degree to which a subject matches the behavioral syndromes, which is usually characterized by poor communicative abilities, peculiarities in social and cognitive capacities and unadaptive behavioral patterns.

The patient in the present example is a male child 9 years old, weighs approximately 60 pounds, and is characterized by the symptoms of deficiencies in short term memory, inadequate independent problem solving skills for his age, deficient spontaneous verbalizations, utterances of diminished length, infrequent request to interact with family members and friends, insufficient capacity to master the complexity of play schemes for his age, and a tendency toward abnormal emotional responses (i.e. laughing when someone is seriously hurt in a movie, not offering assistance to family members when hurt, and not laughing along with siblings while watching cartoons, but instead tensing and becoming overly excited).

In the first vitamin A study in 1996, a sample of the autistic subjects blood was analyzed for retinoic acid content on the belief that there was a deficiency of retinoic acid. At this time, the subject had been receiving up to about 50,000 IU dose of vitamin A palmitate, wherein day 1-0, day 2=25,000 IU and day 3 through day 7=50,000 IU. The RDA is 5,000 IU per day. Upon analysis, it was found that no retinoic acid could be detected in his bloodstream.

In a second study conducted in a period from 1999 to 2000, the autistic subject was orally given retinoic acid in an amount of 20 mg every other day for two weeks, and thereafter the dosage was increased to 40 mg per day for a period up to 5 months. Upon analysis of blood samples analyzed for retinoic acid content, it was found that his blood samples contained no retinoic acid. Although there are numerous cis-trans isomers of vitamin A, there is no possibility for cis-trans isomerism about the cyclohexane double bond because the ring contains only 6 atoms. There are four carbon-carbon double bonds in the chain of atoms attached to the substituted cyclohexane ring, and each has the potential for cis-trans isomerism. Therefore, there are 2×2×2×2, or 16, possible cis-trans isomers for vitamin A.

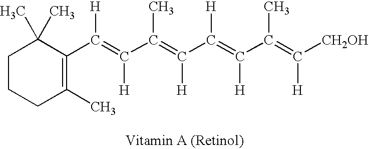

The 9 year old subject weighing approximately 60 pounds was given a dosage of 13-cis retinoic acid in amounts from about 20 mg per day to about 40 mg per 60 lbs of body weight per day. It was found that the optimal dose to provide the best beneficial results of lessening significant amounts of the symptoms of autism was at about 20 mg per 60 lbs of body weight every other day for two weeks, and thereafter the dosage was increased to 40 mg per day. Improvements in lessening the symptoms associated with the autism disorder were noted as early as 10 days into the treatment, with solid gains noted by the 17^{th} day into the treatment.

In addition to an increase in synaptic activity recorded by audio evoked potentials (AEP), it was observed that the symptoms that have been either relieved or improved were:

Improvement in short term memory (i.e. he could remember where he was going without a visual aid), improvement in independent problem solving skills, an increase in spontaneous verbalizations, an increase in the length of utterances, an increase in the frequency of request to interact with family members and friends, an increase in the capability to master the complexity of play schemes, and improvement in tendencies towards more normalized emotional responses (i.e. not laughing when someone is' seriously hurt in a movie, offering assistance to family members when they are hurt, laughing along with sibling who are watching cartoons instead of tensing and becoming overly excited).

The study on this 9 year old male subject also showed that, with higher dosages in excess of 20 mg daily, side effects became apparent. These side effects were dry skin, development of acne and cracking of the lips.

It is known that retinoic acid is the physiological metabolite of retinol, and occurs primarily as the all-trans form, which is shown by the following formula:

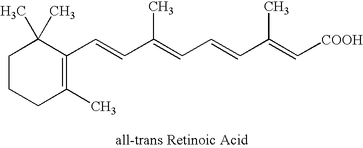

It is the absence of retinoic acid in this form that is believed, upon testing the autistic subject after consumption of Vitamin A (Vitamin Al and Vitamin A₂) that establishes inability to metabolize the Vitamin A.

The Vitamin A acids and analogous compounds, whether all-trans vitamin A acid or 13-cis-Vitamin A acid are represented by the following:

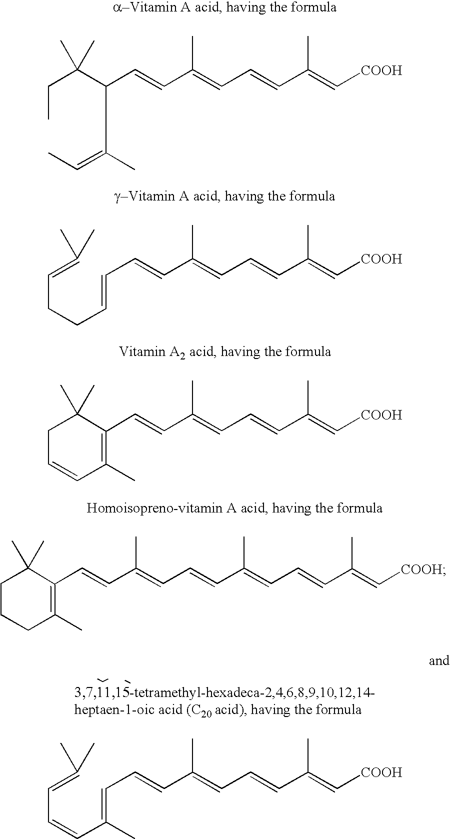

While not wishing to be bound by any theory as to why these physiological metabolites of retinol, known as retinoic acid relieves or lessens symptoms of autism disorder when administered in accordance with the regimen of the invention, it is nevertheless believed that subjects with autistic disorders lack the ability to metabolize retinol, and that, when the metabolite of retinol is properly administered in the forms of retinoic acid, therapeutic benefits are imparted in varying degrees to autistic subjects.

While any of the forms of retinoic acid will work in the context of the invention, the 13-cis-retinoic acid is preferred.

As an example the improvements in relief or elimination of symptoms of autistic disorder after about 17 days of administering 13-cis retinoic acid at dosage levels of from about 20 mg every other day to about 40 mg per day, auditory evoked potential (AEP) responses were given before administering the mentioned dosage and after administration of the dosage regimen. More particularly, the brain stem evoked potential studies showed abnormal responses to frequency modulations in sound prior to administering the dosages of 13-cis retinoic acid, and this possibly suggests involvement of the temporal lobes and thalamocortical afferents. However, after administering the recommended dosage regimen of 13-cis retinoic acid for a period of between about 10 and 17 days, there was a noted improvement in language and cognitive functions that suggested behavioral changes from the lower functioning autistic subject.

The retinoic acid may be prepared in premeasured oral dose units that may comprise tablets or capsules, and each tablet or each capsule may contain an amount of the retinoic acid convenient for administration. For example, the retinoic acid may be incorporated in the form of a dry powder in gelatin capsules to provide a convenient dose unit. Alternatively, the retinoic acid may be administered in the form of a dry powder that is tabletted, and for this purpose may be combined with a tabletting sugar or a diluent such as lactose.

I claim:

1. A method of treating a patient with autism to lessen or alleviate associated autistic symptoms of deficient short term memory, inadequate independent problem solving skills, deficient spontaneous verbalizations, utterances of diminished length, infrequent request to interact with family members and friends, insufficient capacity to master complexity of play schemes, and a tendency toward abnormal emotional responses, comprising:
administering to said patient a therapeutically effective amount of retinoic acid to lessen or alleviate said symptoms.

2. The method of claim 1 wherein said administering is oral.

3. The method of claim 2 wherein said retinoic acid is selected from the group consisting of all-trans vitamin A acid, 13-cis vitamin A acid and mixtures thereof.

4. The method of claim 3 wherein said all-trans vitamin A acid and 13-cis vitamin A are represented by the formulas:

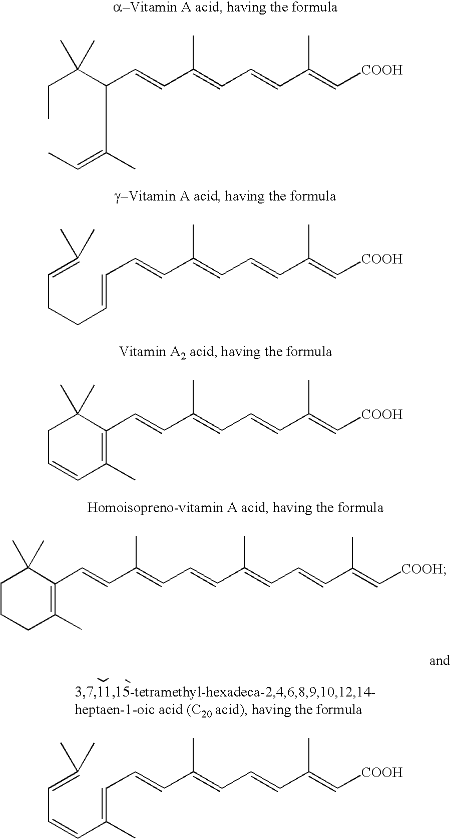

5. The method of claim 4 wherein said retinoic acid is 13-cis vitamin A acid.

6. The method of claim 5 wherein said therapeutically effective amount is from about 20 mg per day to about 40 mg per day.

7. The method of claim 5 wherein said therapeutically effective amount is about 20 mg per 60 lbs of body weight every other day or per 48 hours.

8. The method of claim 6 wherein said therapeutically effective amount is about 20 mg per 60 lbs of body weight per day.

9. The method of claim 6 wherein said therapeutically effective amount is about 30 mg per 60 lbs of body weight per day.

10. The method of claim 6 wherein said therapeutically effective amount is about 40 mg per 60 lbs of body weight per day.
